(19) United States
(12) Patent Application Publication
(10) Pub. No.: US 2020/0114020 A1
(43) Pub. Date: Apr. 16, 2020

(54) ANTIVIRAL COMPOSITION AND APPLICATIONS OF IRON-DOPED APATITE NANOPARTICLES

(71) Applicants: Jessica M. Gregory, Butte, MT (US); Jack L. Skinner, Butte, MT (US); Marisa L. Pedulla, Butte, MT (US); M. Katie Hailer, Butte, MT (US)

(72) Inventors: Jessica M. Gregory, Butte, MT (US); Jack L. Skinner, Butte, MT (US); Marisa L. Pedulla, Butte, MT (US); M. Katie Hailer, Butte, MT (US)

(21) Appl. No.: 16/706,419

(22) Filed: Dec. 6, 2019

Related U.S. Application Data
(63) Continuation-in-part of application No. 15/902,272, filed on Feb. 22, 2018, now Pat. No. 10,532,070.

(51) Int. Cl. A61K 47/69; A61K 9/51; A61K 33/26; A61P 31/22; A61K 47/54

(52) U.S. Cl. CPC A61K 47/6929; A61K 9/5115; A61K 47/542; A61P 31/22; A61K 33/26

(57) ABSTRACT

Iron-doped apatite nanoparticles (IDANPs) are useful for the prevention, treatment, or alleviation of signs or symptoms associated with viral activation or infection. IDANPs have demonstrated a significant influence over herpes simplex virus 1 (HSV-1) infection of two mammalian cell lines. Specifically, IDANPs decreased HSV-1 infection of African Green Monkey kidney epithelial (Vero) cells by 84% and HSV-1 infection of human lung bronchus (BEAS-2B) cells by 71%. In a mouse model, IDANPs delivered at various concentrations and by multiple delivery media, prevented redness, swelling, and/or sores caused by HSV-1 infection in 100% of mice tested during the treatment period. Further, once IDANP treatment had ceased, mice did not experience redness, swelling, and/or sores for at least one and up to nine days thereafter, demonstrating IDANPs not only prevent signs and symptoms during treatment, but that IDANPs prevent future signs and symptoms caused by mammalian viral infections. IDANPs consist of hydroxyapatite (HA) doped with iron. HA is a mineral known to be biocompatible and analogous to the inorganic constituent of mammalian bone and teeth and has been approved by the Food and Drug Administration (FDA) for many applications in medicine and dentistry. Lactate Dehydrogenase (LDH) and XTT (2,3-Bis 2-methoxy-4-nitro-5-sulfophenyl-2H-tetrazolium-5-carboxanilide inner salt) cytotoxicity assays revealed that IDANPs are largely non-toxic to Vero, BEAS-2B, and human cervical cancer (HeLa) cells lines. HSV-1 afflicted individuals in the United States have been estimated as high as ⅔ the population. Because IDANPs dramatically decrease HSV-1 infection and are largely non-toxic, their application as an antiviral agent is evident. Further, although iron(III) alone has been shown to diminish replication of DNA and RNA viruses, IDANP cytotoxicity studies indicate that encasement and delivery of iron within an apatite unit cell structure diminishes significantly, and in some cases eliminates, cytotoxicity posed by the introduction of iron(III) alone.

### CROSS-REFERENCE TO RELATED APPLICATIONS

This is a continuation-in-part of U.S. Non-Provisional application Ser. No. 15/902,272, filed on Feb. 22, 2018, now pending, the disclosure of which is hereby incorporated by reference in its entirety to provide continuity of disclosure.

### STATEMENT REGARDING FEDERALLY SPONSORED RESEARCH OR DEVELOPMENT

Not applicable.

### THE NAMES OF THE PARTIES TO A JOINT RESEARCH AGREEMENT

Not applicable.

### REFERENCE TO SEQUENCE LISTING, A TABLE, OR A COMPUTER PROGRAM LISTING COMPACT DISC APPENDIX

Not applicable.

### BACKGROUND OF THE INVENTION

Compositions of Iron-Doped Apatite Nanoparticles (IDANP's) herein described, dramatically decrease HSV-1 infection, and are largely non-toxic. As such, IDANP's are useful for the prevention, treatment, or alleviation of signs or symptoms associated with viral activation or infection. The nanoparticles (NPs) used in this research are composed of hydroxyapatite (HA) Ca₁₀(PO₄)₆(OH)₂, doped with iron. HA, which is a constituent of mammalian bones and teeth, has been extensively studied and approved by the Food and Drug Administration (FDA) for applications in medicine and dentistry (Palmer et al., Chemical Reviews, 2008 & Hench, Journal of the American Ceramic Society, 1998).

In a publication by Felix d'Herelle in 1931, viruses which specifically kill bacteria were used to treat acute bacterial infection (d'Herelle, Bulletin of the New York Academy of Medicine, 1931). These viruses were termed bacteriophage (phage) by d'Herelle, and treatment of bacterial infection by phage has since been referred to as phage therapy. However, the discovery and use of traditional antibiotics such as penicillin (Fleming, British Journal of Experimental Pathology, 1929) de-emphasized wide spread use of phage therapy. In 2013, the Centers for Disease Control and Prevention estimated that each year, 2 million people in the United States become infected with antibiotic-resistant bacteria, of which, approximately 23,000 die as a direct result of such infections (2013). Rapid bacterial resistance to traditional antibiotics therefore calls for alternative therapies such as phage therapy to be revisited. Previous research has shown that addition of IDANPs to bacteria prior to phage exposure results in increased bacterial plaques in vitro (Andriolo et al., Journal of Vacuum Science and Technology B, 2013). Because IDANPs enhance phage killing of bacteria, initial interest in their study as a adjuvant to phage therapy was garnered.

Bacterial viruses (phage) and human viruses have many similarities including structure and mechanism of infection. To ensure safety of IDANPs in a human system, it had to be established that while these nanoparticles (NPs) increased phage infection and killing of bacterial cells, that IDANPs did not also increase eukaryotic virus infections and killing of eukaryotic cells. To test IDANP-effect on eukaryotic virus infection of eukaryotic cells, experiments were carried out using Chlorella variabilis NC64A (NC64A) and its virus, Paramecium bursaria chlorella virus 1 (PBCV-1) (Andriolo et al., IEEE Transactions on Nanobioscience, 2016). Results indicated that in an algal system, viral infections were not increased or decreased by the addition of IDANPs.

Previous work has shown iron(III) inactivates HSV-1 (Sagripanti et al., Applied and Environmental Microbiology, 1993), and in more recent studies, it has been shown that iron(III) inhibits replication of DNA and RNA viruses (Terpilowska et al., Biometals, 2017). However, specific therapeutic applications of IDANPs regarding viral infection have previously not been disclosed or studied. The IDANP compositions and methods of use herein described, dramatically decrease viral infection, and are largely non-toxic to mammalian cells. As such, IDANP's are useful for the prevention, treatment, or alleviation of signs or symptoms associated with viral activation or infection.

### BRIEF SUMMARY OF THE INVENTION

IDANP effect on viral infection of African Green Monkey kidney epithelial cells (Vero) and human lung bronchus cells (BEAS-2B) was investigated. The virus used for testing was herpes simplex virus 1 (HSV-1). Investigations revealed that IDANP influence over HSV-1 infection of Vero and BEAS-2B cells was significant (p<0.001). IDANPs decreased HSV-1 infection of Vero cells by 84%, and HSV-1 infection of BEAS-2B cells by 71%.

IDANPs used in this research are composed of HA doped with iron. HA is a constituent of mammalian bones and teeth and has been extensively studied and approved for medical and dental applications by the FDA (Palmer et al., Chemical Reviews, 2008 & Hench, Journal of the American Ceramic Society, 1998). To establish the doping of HA with iron would not diminish biocompatibility, cytotoxicity evaluations were performed on IDANPs. The first cytotoxicity test used measured cell distress by lactate dehydrogenase (LDH) release, and the second, measured cell health by oxidoreductase enzyme activity. Results showed minimal to no increase in LDH release by three cell lines: 0.00% in BEAS-2B and human cervical cancer (HeLa) cell lines, and 4.27% in Vero cell line. IDANP effect on cell health was evaluated by XTT (2,3-Bis 2-methoxy-4-nitro-5-sulfophenyl-2H-tetrazolium-5-carboxanilide inner salt) cytotoxicity assay. XTT assays revealed no significant difference in oxidoreductase enzyme activity in Vero (p=0.276), BEAS-2B (p=0.131), or HeLa (p=0.960) cell lines. An alternative test to XTT cytotoxicity assay is an MTT (3-(4,5-dimethylthiazol-2-yl)-2,5-diphenyltetrazolium bromide) cytotoxicity assay. Both tests involve the reduction of tetrazolium salts (XTT or MTT) to formazan by oxidoreductase enzymes released by cells and are good indicators of cell health. Previous researchers showed that iron(III) ions alone inhibit replication of DNA and RNA viruses (Terpilowska et al., Biometals, 2017). However, using the MTT assay, these researchers also showed that 150 μM iron(III) (as is found in IDANPs), caused a reduction in cell viability of HEp-2 (HeLa contaminant) cells to ˜73%. For comparison, at the same concentration, IDANPs maintain 100% cell viability in Vero and HeLa cell lines, and 94.6% in the BEAS-2B cell line. Therefore, IDANPs provide a biocompatible method for iron delivery to act as an effective and safe anti-viral agent.

IDANPs are synthesized using wet chemical precipitation methods (Andriolo et al., Journal of Vacuum Science and Technology B, 2013 & Andriolo et al., IEEE Transactions on Nanobioscience, 2016). Synthesis of IDANPs involves iron replacement of calcium in the apatite unit cell to 30% iron in the molar ratio of total iron plus calcium. Citrate was used as a capping agent to arrest NPs at the nanoscale. The reaction formula is as follows:

7 Ca(OH)2+3FeCl3+6KH2PO4 Citric Acid Ca7Fe3(PO4)6(OH)2+6KOH+12H2O+9Cl—

During synthesis of 30% IDANPs with 1× citrate, a 500 mL flask held at 25° C. was filled with 200 mL deionized water and stirred by stir bar as the following reagents were added in the order listed:
0.260 g Calcium Hydroxide (Ca(OH)2)
0.243 g Iron Chloride (FeCl3)
0.263 g Citric Acid Anhydrous (C6H7O7)
0.408 g monopotassium phosphate (KH2PO4) that was pre-dissolved in 50 mL deionized water is added dropwise over a period of 1 minute.

The final solution was measured at a pH of approximately 4.5 and brought up to a pH of 7.5 using 1 M NaOH. IDANPs were then stirred at 25° C. for seven days. After seven days, IDANPs were centrifuged for 30 min at 2000 rpm. IDANP supernatant was then removed, leaving the IDANP pellet. The pellet was washed 2× with sterile, deionized water (18 Me), and IDANPs were re-suspended in deionized water before being sterilized in an autoclave for 40 minutes. IDANP concentration resulting from this synthesis procedure was estimated to be 1.54 mg/mL by simple drying method and weighing of dried IDANPs.

Original cell cultures were maintained at 37° C. and 5% carbon dioxide in 75 cm2 flasks. Original cultures were grown in minimal essential media (MEM) with 10% fetal bovine serum (FBS) and 1% penicillin-streptomycin (P-S) antibiotic.

To evaluate IDANP influence over HSV-1 infection of Vero and BEAS-2B cells, plaque assays were used. HSV-1 maintenance and storage and plaque assay methods were adapted from (Blaho et al., Current Protocols in Microbiology, 2005). When original cultures were confluent, Vero and BEAS-2B cells maintained in 75 cm² flasks were lifted with trypsin (0.25%)-EDTA and split into 25 cm² flasks (15-17,000 cells per flask counted directly by hemocytometer) for plaque assay. In 25 cm² flasks, Vero and BEAS-2B cells were grown in MEM with 5% FBS and 1% P-S for 3-4 days, or until confluent. On the day of plaque assay, HSV-1 was removed from −80° C. freezer and thawed in the biosafety hood. Once thawed completely, HSV-1 was diluted into 199V media (Blaho et al., Current Protocols in Microbiology, 2005), or 199V with suspended IDANPs (1.54 mg/mL) to a pre-determined concentration for countable plaques (˜50-100 PFUs/mL). To prepare 199V media with suspended IDANPs (199V^{NP}), IDANPs were centrifuged and supernatant removed before IDANPs were re-suspended in 199V. Growth media was then aspirated from 25 cm² flasks and replaced with 1 mL of HSV-1 in 199V or HSV-1 in 199VNP. HSV-1 was allowed to adsorb to the cells for 2 hr in an incubator (37° C., 5% CO2) with gentle rocking every 30 min. After the 2 hr adsorption period, 199V or 199V^{NP} was removed from the flasks and replaced with 3 mL MEM with 7.5 μg/mL pooled human immunoglobulin (IgG). Flasks were then incubated for 3 days at 37° C. with 5% CO2. After 3 days, media was removed from the 25 cm² flasks. To each flask, 1 mL methanol was added 5 min. After 5 min, methanol was removed from the flasks and replaced with 2 mL KaryoMax Giemsa Stain (diluted 1:10 with distilled water). Cell monolayers were stained for 20 min before the stain was removed and cell monolayers were rinsed with deionized water. Plaques were subsequently enumerated. Plaque assay was repeated in three separate experiments, with four, five, and seven pseudo-replicates per treatment condition for Vero cell line, and three, six, and seven for the BEAS-2B cell line. Negative controls were performed in all experiments, in which 199V or 199VNP without HSV-1 were exposed to the cell lines during the 2 hr infection period.

For LDH cytotoxicity assay, original cultures of Vero, BEAS-2B, or HeLa were subject to lifting by 5 mL trypsin (0.25%)-EDTA before being pelleted by centrifugation (3000 rpm, 5 min) and trypsin subsequently replaced with MEM (10% FBS, 1% P-S). Cells were counted directly by hemocytometer and plated in a 96-well plate (7,000-9,000 cells per well) in 100 μL MEM (10% FBS, 1% P-S) and grown to confluence over the next 1-2 days. After cells were confluent, MEM was removed from the wells and replaced with either: (1) fresh MEM (10 wells), (2) fresh MEM with suspended IDANPs (25° C., 30% Fe, 5.5 mM citrate at 1.54 mg/mL, 10 flasks), or Dulbecco's phosphate buffered saline (DPBS, two flasks) as a vehicle control. Treatments were applied to the cell monolayers for 24 hr. Approximately 1 hr before the end of treatment time, cytotoxicity kit reagents were prepared according to protocols distributed by Biovision for Colorimetric Assay II (Documentation can be found at https://www.biovision.com/documentation/datasheets/K313.pdf). Approximately 15 min before the end of treatment time, 10 μL of prepared cell lysis solution was added to five of the MEM-only wells and pipetted up and down. The five wells inoculated with cell lysis solution constituted the high control for LDH cytotoxicity evaluation. After treatment time, solution from each of the wells was collected and placed in microcentrifuge tubes. Microcentrifuge tubes were spun at 600 rgf for 5 min to pellet any large cell components and IDANPs. After centrifugation, 10 μL from each microcentrifuge tube was transferred to individual wells in a fresh 96-well plate. To each well, 100 μL LDH Reaction Mix was added, pipetted up and down to mix, and incubated (37° C.) in the UV-Vis spectrometer for 1 hr 15 min with constant monitoring every 3 min at 450 nm. Biovision protocols indicated the reading should be taken when the high control was ˜2.0 and the low control was <0.8. Wells with cell monolayers treated with NPs were repeated in 10 wells, low and high controls were repeated in five replicate wells each, and vehicle controls were performed in duplicate. Reference was taken after analysis period by taking additional readings at 450 nm and 620 nm simultaneously. Cytotoxicity (%) was determined by the following formula provided by Biovision (Documentation can be found at https://wwvv.biovision.com/documentation/datasheets/K313.pdf):

Cytotoxicity (%)=[((Test Sample−Low Control))/((High Control−Low Control))]×100%

Using cytotoxicity (%), it was determined that IDANPs posed 0.00% cytotoxicity to BEAS-2B and HeLa cell lines, and 4.27% cytotoxicity to the Vero cell line.

For XTT cytotoxicity evaluation, original cultures of Vero, BEAS-2B, and HeLa were subject to 5 mL trypsin (0.25%)-EDTA before being pelleted by centrifugation (3000 rpm, 5 min), and trypsin was subsequently replaced with MEM (10% FBS, 1% P-S). Cells were counted directly by hemocytometer and plated in a 96-well plate (7,000-9,000 cells per well) in 100 μL, =MEM (10% FBS, 1% P-S) and grown to confluence over the next 1-2 days. After cells were confluent, MEM was removed from the wells and replaced with either: (1) fresh MEM (11 wells), (2) fresh MEM with suspended IDANPs (25° C., 30% Fe, 5.5 mM citrate at 1.54 mg/mL, 11 flasks), Dulbecco's phosphate buffered saline (DPBS, 2 flasks) as a vehicle control. Treatments were applied to cell monolayers for 24 hr. During the last hour of exposure to the treatments, XTT was dissolved 0.01 g into 10 mL DPBS, and phenazine methosulfate (PMS) was dissolved 0.05 g into 1 mL sterile, deionized water (18MΩ). Then, 100 μL PMS solution was pipetted into 5 mL of the XTT solution. During preparation, all of these solutions were kept on ice. In addition, 10 μL cell lysis solution was added to one well containing MEM only, and one well containing MEM with suspended IDANPs as dead controls. At the conclusion of treatment, all solution was removed from the wells, cell monolayers were washed 2× with DPBS, and replaced with fresh MEM. This wash/replacement procedure was used to eliminate any signal coming from XTT interacting with IDANPs or IDANPs alone. Each well was subsequently inoculated with 100 μl XTT/PMS solution, and plates were placed back into the incubator for 2 hrs, with final absorption read at 450 nm (reference at 620 nm). During these studies, 10 replicate wells were treated with MEM only, ten with MEM with suspended IDANPs, two dead controls (one per MEM or MEM with IDANPs) were used, as well as two vehicle controls (treated with DPBS). Using a one way ANOVA in SigmaPlot (V.11) to analyze XTT results, no significant difference in mitochondrial enzyme activity was observed between Vero, BEAS-2B, or HeLa cells which had or had not been exposed to IDANPs.

Where necessary, significance of results were determined in SigmaPlot using a one way ANOVA V.11.

IDANPs have demonstrated a significant influence over HSV-1 infection of two mammalian cell lines. Specifically, IDANPs decreased HSV-1 infection of Vero cells by 84% and HSV-1 infection of BEAS-2B cells by 71%. Lactate Dehydrogenase (LDH) and XTT cytotoxicity assays revealed that IDANPs are largely non-toxic to Vero, BEAS-2B, and HeLa cells lines. Because IDANPs dramatically decrease HSV-1 infection and are largely non-toxic, their application as an antiviral agent is evident. Further, although iron(III) alone has been shown to diminish replication of DNA and RNA viruses (Terpilowska et al., Biometals, 2017), IDANP cytotoxicity studies indicate that encasement and delivery of iron within an apatite unit cell structure diminishes significantly, and in some cases eliminates, cytotoxicity posed by the introduction of iron(III) alone. As such, compositions and methods of using IDANPs for the prevention, treatment, or alleviation of signs or symptoms associated with viral activation or infection are disclosed herein. The compositions of this invention include IDANPs suspended in one of the following, but are not limited to the following: solid, semi-solid, Newtonian or Non-Newtonian fluid, or powder. One skilled in the art would recognize that such compositions could be delivered by various therapeutic means including, but not limited to injection, oral administration, or direct application.

To demonstrate feasibility of IDANPs to not only prevent viral infection and replication, but to also prevent signs and symptoms associated with viral infection and replication, IDANP formulations were tested in vivo in mice at the Animal Resource Center (ARC) at Montana State University (MSU). Protocols and euthanasia stipulations and methods were pre-approved by the biosafety and institutional animal care and use committees at ARC at MSU. To prepare treatments, IDANPs were mixed by hand into three different commercial lip products (CLPs) at three different concentrations (0.8 mg/mL, 1.6 mg/mL, and 3.2 mg/mL). Mice were infected with HSV-1 by a simple abrading of the lip epithelium and painting on of HSV-1. Mice were thereafter treated three times per day with treatment for five days and observed post-treatment. After mice exhibited significant swelling, redness, or lesion (cold sore) formation, they were humanely euthanized. The following treatments were each given to three mice so that statistical significance of the results were valid: (1) CLP 1+0.8 mg/mL IDANPs, (2) CLP 1+0.16 mg/mL IDANPs, (3) CLP 1+3.2 mg/mL IDANPs, (4) CLP 2+0.8 mg/mL IDANPs, (5) CLP 2+0.16 mg/mL IDANPs, (6) CLP 2+3.2 mg/mL IDANPs, (7) CLP 3+0.8 mg/mL IDANPs, (8) CLP 3+0.16 mg/mL IDANPs, (9) CLP 3+3.2 mg/mL IDANPs, (10) CLP 1, (11) CLP 2, (12) CLP 3. In addition, treatments (11), (12), and (13) were also given to three mice each, who were not infected with HSV-1 to serve as negative controls. One of the CLPs used stained the mice's lips to the extent that signs or symptoms could not be visualized easily, and therefore these mice were not considered in our results. All mice infected with HSV-1 and treated with CLPs NOT containing IDANPs exhibited lesion formation three days after being infected with HSV-1 and were euthanized. All mice infected with HSV-1 and treated with one of the two CLPs CONTAINING ANY CONCENTRATION of IDANPs did not exhibit swelling, redness, or lesion formation which warranted euthanasia for the five-day treatment period, plus one day where the mice were not treated. Most mice were euthanized on the eighth day post-HSV-1 infection (three days after treatment ceased). However, one mouse did not exhibit symptoms until the ninth day (four days post infection), and one mouse did not exhibit symptoms until day fourteen (nine days post infection). Results of this study demonstrate that IDANPs not only prevent signs and symptoms associated with viral infection during treatment, but that IDANP treatment prevents future signs and symptoms associated with viral infection even after treatment has ceased.

IDANP compositions used for prevention, treatment, or alleviation of signs or symptoms associated with viral activation or infection are prepared as follows: IDANPs are synthesized as stated herein (see [0007] and [0026]). Supernatant created during IDANP synthesis/preparation is removed by some method, for example, centrifugation of the particles and removal of supernatant by pipette. IDANPs without supernatant are then suspended by any means suitable into any solid, semi-solid, Newtonian or Non-Newtonian fluid, or powder by mixing. One example would include simply mixing synthesized IDANPs into these materials by stir bar on a stir plate, or vortexing.

### BRIEF DESCRIPTION OF THE SEVERAL VIEWS OF THE DRAWINGS

FIG. 1 Microscope images of confluent A Vero, B BEAS-2B, and C HeLa cells. Cells were grown in at 5% CO₂ in 75 cm² flasks containing 23-25 mL MEM supplemented with 10% FBS and 1% P-S. To maintain healthy cultures, cell media was changed every 3-4 days, and confluent cultures were lifted with trypsin (0.25%)-EDTA and split by removing all but 1 mL of the original cell culture, before being placed back in the incubator with fresh media.

FIG. 2 Electron micrographs of IDANPs synthesized under different conditions. A Scanning electron micrograph of IDANPs synthesized at 25° C., with 30% iron-doping, and 5.5 mM citrate. IDANPs show spherical morphology with particle diameters ranging from 20-50 nm. B Transmission electron micrograph showing IDANPs synthesized at 25° C., with no iron or citrate. Apatite nanoparticles made in this way revealed shard or broken glass-like morphology. C Scanning electron micrograph of IDANPs synthesized at 25° C., with 30% iron-doping, and no citrate. Lack of citrate during preparation resulted in IDANP elongation.

FIG. 3 Pictures showing A IDANPs synthesized at 25° C., with 30% iron-doping, and 5.5 mM citrate, B ANPs synthesized at 25° C., with 0% iron-doping, and 5.5 mM citrate.

FIG. 4 IDANPs synthesized at 25° C. with 30% iron-doping and 5.5 mM citrate. During synthesis, iron incorporation turns the colloid solution orange. A Picture of bulk IDANPs after synthesis. B Scanning electron micrograph showing IDANPs range from 20-50 nm in diameter and are approximately spherical (although amorphous) in shape.

FIG. 5 Plaque assay results from a single experiment using Vero cells. Flasks on the top row (with exception of the far-right flask) were infected with HSV-1 in 199V media. Flasks on the bottom row (with exception of the far-right flask) were infected with HSV-1 in 199V^{NP} media. Results of three plaque assay experiments each containing four to seven pseudo-replicates per treatment showed an average decrease in HSV-1 infection of Vero cell monolayers of 84% when HSV-1 infection was carried out in 199V media with suspended IDANPs as compared to 199V alone (p<0.001).

FIG. 6 Plaque assay results from a single experiment using BEAS-2B cells. Flasks on the top row (with exception of the far-right flask) were infected with HSV-1 in 199V media. Flasks on the bottom row (with exception of the far-right flask) were infected with HSV-1 in 199V^{NP} media. Results of three plaque assay experiments each containing 3-7 pseudo-replicates per treatment showed an average decrease in HSV-1 infection of BEAS-2B cell monolayers of 71% when HSV-1 infection was carried out in 199V media with suspended IDANPs (199V^{NP}) as compared to 199V alone.

FIG. 7 Plaque assay results showing IDANPs diluted 10× (to 0.154 mg/mL) in 199V media still influence a 28% decrease in HSV-1 infection of BEAS-2B cell monolayers. IDANPs diluted beyond 12× or more did not affect HSV-1 infection of BEAS-2B significantly as compared to the control.

FIG. 8 Plaque assay results standardized from three plaque assay experiments using Vero cells. When HSV-1 infection was carried out in 199V^{NP} media, an 84% reduction of infection was observed, as compared to 199V media alone (p<0.001).

FIG. 9 Plaque assay results standardized from three plaque assay experiments using BEAS-2B cells. When HSV-1 infection was carried out in 199V^{NP} media, a 71% reduction of infection was observed, as compared to 199V media alone (p<0.001).

FIG. 10 Results from XTT cytotoxicity assays performed with MEM versus MEM with suspended IDANPs. Results show that in Vero (p=0.276), BEAS-2B (0.131), and HeLa (p=0.960) cell lines, no significant decrease in enzyme activity is imposed when cell monolayers are exposed to IDANPs for 24 hr. Statistics were evaluated in SigmaPlot using a one-way ANOVA (V.11). Error bars extend one standard deviation above and below the mean.

FIG. 11 Experimental setup used to determine feasibility of IDANPs as a treatment for the prevention of signs and symptoms associated with mammalian viral infection. The experimental design included three different delivery mediums, each containing one of three different IDANP concentrations or no IDANPs. Nine positive control mice infected with HSV-1 but treated with one of the three CLPs NOT containing IDANPs were used, and nine negative control mice who were not infected with HSV-1 but still treated with CLPs NOT containing IDANPs were used. Treatments were delivered in triplicate to ensure significant results were obtained, positive control mice were used to demonstrate sign/symptom prevention was not due to the delivery medium itself, and negative control mice ensured swelling, redness, or lesion formation were due to our HSV-1 infection process and not some other environmental factor. One of the CLPs used stained the mice's lips to the extent that signs or symptoms could not be visualized easily, and therefore these mice were not considered in the results.

FIG. 12 Graphical representation of mouse studies to test the feasibility of IDANP treatments preventing signs and/or symptoms associated with mammalian viral infection. Results show that mice that were infected with HSV-1 and treated with CLP that did not contain IDANPs exhibited lesions by the third day post-infection. Negative control mice treated with CLP but never infected with virus did not exhibit signs and or symptoms throughout the study. All mice that were infected with HSV-1 but treated with either of two lip products containing one of three concentrations of IDANPs did not develop viral lesions at least one and up to nine days post-treatment.

### DETAILED DESCRIPTION OF THE INVENTION

Virus Maintenance and Cell Culture.

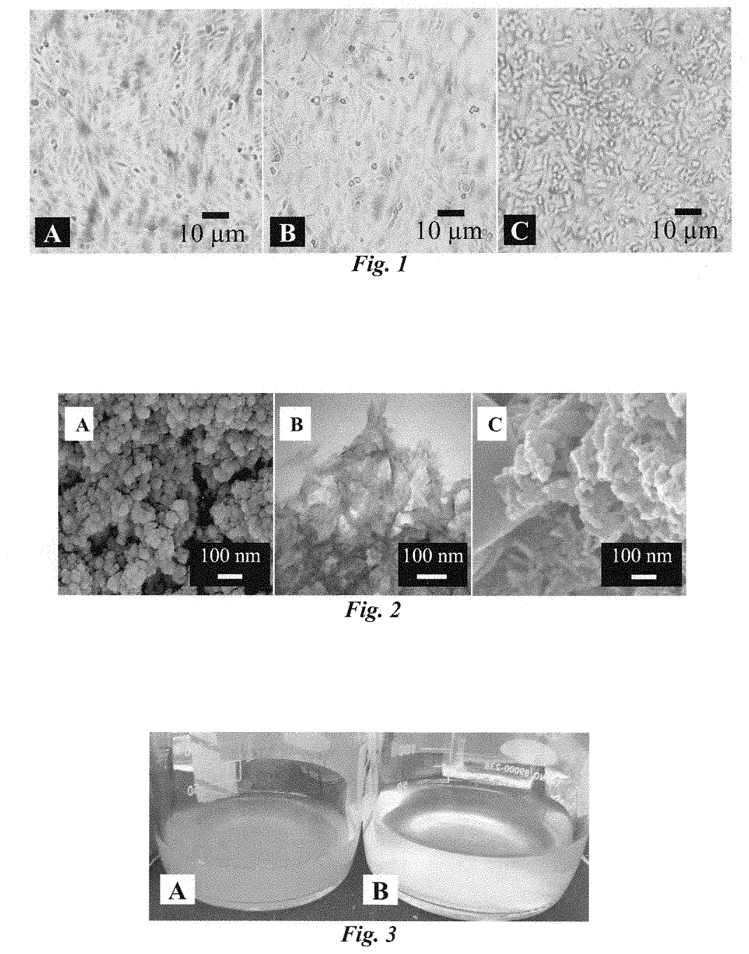

HSV-1 virus stock was prepared and stored as previously described (Blaho et al., Current Protocols in Microbiology, 2005) and thawed (from −80° C.) just prior to being added to Vero or BEAS-2B cells for infection. Vero, BEAS-2B, and HeLa cell lines were maintained in an incubator (7° C., 5% CO₂) in 75 cm² flasks with minimal essential media (MEM) supplemented with 10% fetal bovine serum (FBS) and 1% penicillin-streptomycin antibiotic (P-S). To maintain healthy cultures, cell media was changed every 3-4 days, and confluent cultures were lifted with trypsin (0.25%)-EDTA and split by removing all but 1 mL of the original cell culture, before being placed back in the incubator with fresh media. (FIG. 1).

IDANP Synthesis.

Synthesis of IDANPs has been described previously (Andriolo et al., Journal of Vacuum Science and Technology B, 2013 & Andriolo et al., IEEE Transactions on Nanobioscience, 2016). IDANPs resemble HA, a mineral that is well known to be biocompatible and most analogous to the inorganic constituent of mammalian bone and teeth (Palmer et al., Chemical Reviews, 2008). Such properties allow these NPs to serve as biocompatible adjuvants capable of entering a physiological system without significant immune system rejection. Previous synthesis investigations have accomplished the synthesis of citrate functionalized and/or dispersed IDANPs (FIG. 2A). The citrate ions complex with Ca²⁺, and mediate the reaction leading to the formation of nanoapatite particles. The carboxylates of citrate, which at physiological pH are deprotonated, give rise to Coulombic repulsion in adjacent NPs. This repulsion causes dispersion and colloid formation. During synthesis, it was assumed that iron replaces calcium in the apatite unit cell to 30% iron in the ratio of total iron plus calcium. Lack of iron during synthesis results in elongated glass-like morphology of the particles (FIG. 2B). Citrate was used as a capping agent to arrest NPs at the nanoscale. Lack of citrate results in extended particle growth which resembles longer chains (FIG. 2C). The reaction formula is as follows:

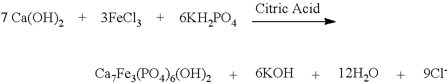

During synthesis of 30% IDANPs with 1× citrate, a 500 mL flask held at 25° C. was filled with 200 mL deionized water and stirred by stir bar as the following reagents were added in the order listed:
0.260 g Calcium Hydroxide (Ca(OH)₂)
0.243 g Iron Chloride (FeCl₃)
0.263 g Citric Acid Anhydrous (C₆H₇O₇)
0.408 g monopotassium phosphate (KH₂PO₄) that was pre-dissolved in 50 mL deionized water is added dropwise over a period of 1 minute.
   The final solution was measured at a pH of approximately 4.5 and brought up to a pH of 7.5 using 1 M NaOH. IDANPs were then stirred at 25° C. for seven days. After seven days, IDANPs were centrifuged for 30 min at 2000 rpm. IDANP supernatant was then removed, leaving the IDANP pellet. The pellet was washed 2× with sterile, deionized water (18 MΩ), and IDANPs were re-suspended in deionized water before being sterilized in an autoclave for 40 minutes. IDANP concentration resulting from this synthesis procedure was estimated to be 1.54 mg/mL by simple drying method and weighing of dried IDANPs. The addition of iron results in the bulk colloid solution appearing orange in color (FIG. 3, FIG. 4A). IDANPs are approximately 20-50 nm in diameter once synthesis is completed (FIG. 4B).

Plaque Assay.

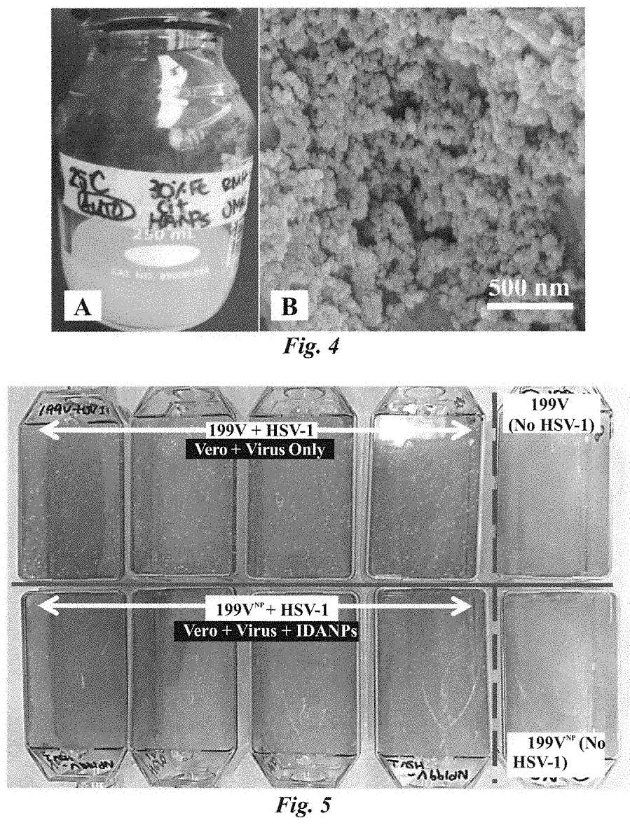

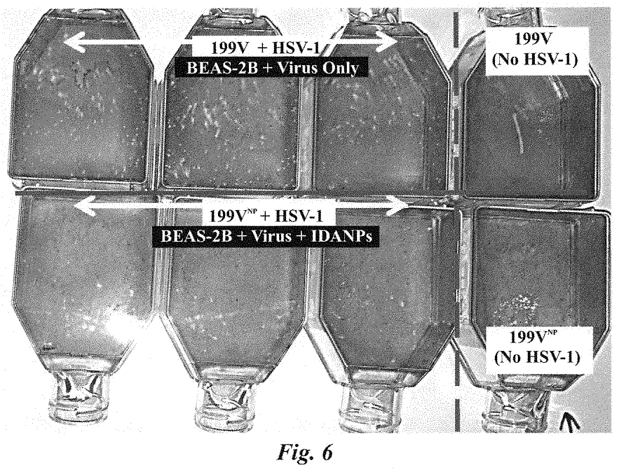

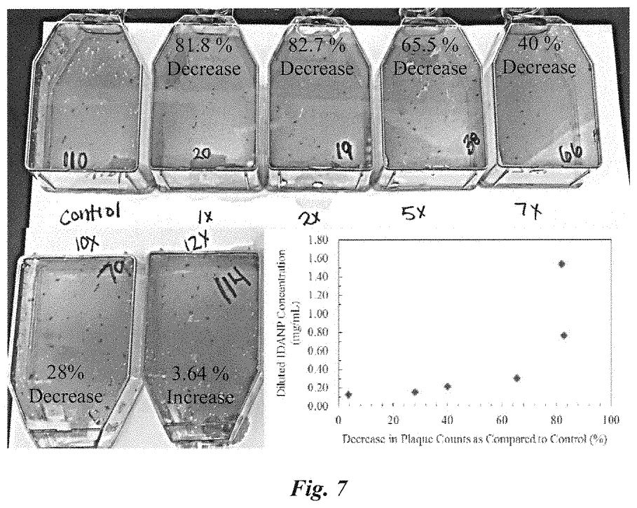

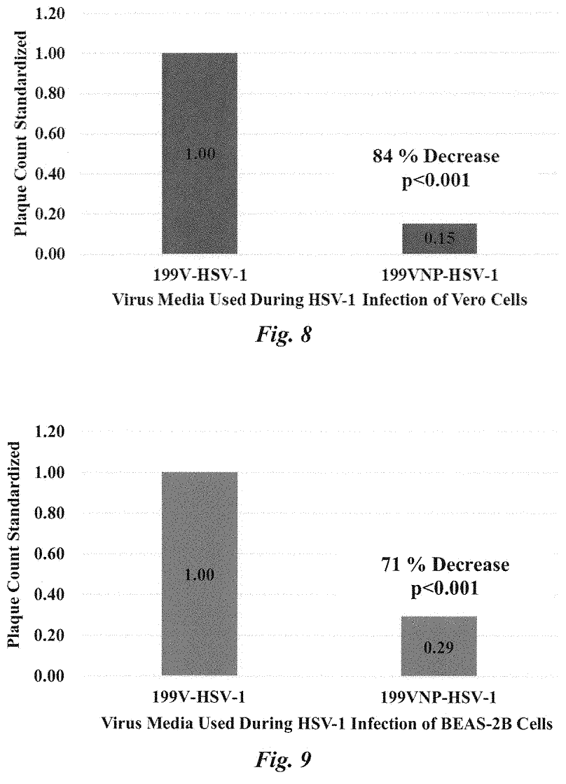

Plaque assay methods were adapted and modified from (Blaho et al., Current Protocols in Microbiology, 2005). When confluent, Vero or BEAS-2B cells maintained in 75 cm² flasks were lifted with trypsin (0.25%)-EDTA and split into 25 cm² flasks (15-17,000 cells per flask counted directly by hemocytometer) for plaque assay. In 25 cm² flasks, Vero and BEAS-2B cells were grown in MEM with 5% FBS and 1% P-S for 3-4 days, or until confluent. On the day of plaque assay, HSV-1 was removed from −80° C. freezer, and thawed in the biosafety hood. Once thawed completely, HSV-1 was diluted into 199V media (Blaho et al., Current Protocols in Microbiology, 2005), or 199V with suspended IDANPs (1.54 mg/mL) to a pre-determined concentration for countable plaques (˜50-100 PFUs/mL). To prepare 199Vmedia with suspended IDANPs (199V^{NP}), IDANPs were centrifuged and supernatant removed before IDANPs were re-suspended in 199V. Growth media was then aspirated from 25 cm² flasks and replaced with 1 mL of HSV-1 in 199V or HSV-1 in 199V^{NP}. HSV-1 was allowed to absorb to the cells for 2 hr in an incubator (37° C., 5% CO2) with gentle rocking every 30 min. After the 2 hr. adsorption period, 199V or 199V^{NP} was removed from the flasks and replaced with 3 mL MEM with 7.5 μg/mL pooled human immunoglobulin (IgG). Flasks were then incubated for 3 days at 37° C. with 5% CO₂. After 3 days, media was removed from the 25 cm² flasks. To each flask, 1 mL methanol was added 5 min. After 5 min, methanol was removed from the flasks and replaced with 2 mL KaryoMax Giemsa Stain (diluted 1:10 with distilled water). Cell monolayers were stained for 20 min before the stain was removed and cell monolayers were rinsed with deionized water. Plaques were subsequently enumerated. Plaque assay was repeated in three separate experiments, with four, five, and seven pseudo-replicates per treatment condition for Vero cell line, and three, six, and seven for the BEAS-2B cell line. Negative controls were performed in all experiments, in which 199V or 199V^{NP} without HSV-1 were exposed to the cell lines during the 2 hr infection period. Results of plaque assays showed IDANPs influenced an 84% decrease in HSV-1 infection of Vero cells (FIG. 5), and a 71% decrease in HSV-1 infection of BEAS-2B cells (FIG. 6). Diluted to 10×, IDANPs still influenced a 28% decrease in HSV-1 infection of BEAS-2B cells (FIG. 7). Results of plaque assay experiments were standardized by dividing each 199V^{NP}-HSV-1 treated flask plaque count by the average control (199V-HSV-1) plaque count value for that particular plaque assay (FIG. 8, FIG. 9). Statistics were evaluated in SigmaPlot V.11 using a one-way ANOVA.

LDH Cytotoxicity Assay.

For LDH cytotoxicity assay, Vero, BEAS-2B, or HeLa cells were lifted from 75 cm² flasks with 5 mL trypsin (0.25%)-EDTA before being pelleted by centrifugation (3000 rpm, 5 min) and trypsin subsequently replaced with MEM (10% FBS, 1% P-S). Cells were counted directly by hemocytometer and plated in a 96-well plate (7,000-9,000 cells per well) in 100 μL MEM (10% FBS, 1% P-S) and grown to confluence over the next 1-2 days. After cells were confluent, MEM was removed from the wells and replaced with either: (1) fresh MEM (10 wells), (2) fresh MEM with suspended IDANPs (25° C., 30% Fe, 5.5 mM citrate at 1.54 mg/mL, 10 flasks), or Dulbecco's phosphate buffered saline (DPBS, two flasks) as a vehicle control. Treatments were applied to the cell monolayers for 24 hr. Approximately 1 hr before the end of treatment time, cytotoxicity kit reagents were prepared according to protocols distributed by Biovision for Colorimetric Assay II (Documentation can be found at: https://www.biovision.com/documentation/datasheets/K313.pdf). Approximately 15 min before the end of treatment time, 10 μL of prepared cell lysis solution was added to five of the MEM-only wells and pipetted up and down. The five wells inoculated with cell lysis solution constituted the high control for LDH cytotoxicity evaluation. After treatment time, solution from each of the wells was collected and placed in microcentrifuge tubes. Microcentrifuge tubes were spun at 600 rgf for 5 min to pellet any large cell components and IDANPs. After centrifugation, 10 μL from each microcentrifuge tube was transferred to individual wells in a fresh 96-well plate. To each well, 100 μl LDH Reaction Mix was added, pipetted up and down to mix, and incubated (37° C.) in the UV-Vis spectrometer for 1 hr 15 min with constant monitoring every 3 min at 450 nm. Biovision protocols indicated the reading should be taken when the high control was ˜2.0 and the low control was <0.8. Wells with cell monolayers treated with NPs were repeated in 10 wells, low and high controls were repeated in five replicate wells each, and vehicle controls were performed in duplicate. Reference was taken after analysis period by taking additional readings at 450 nm and 620 nm simultaneously. Cytotoxicity (%) was determined by the following formula provided by Biovision (https://www.biovision.com/documentation/datasheets/K313.pdf):

$$Cytotoxicity ( \% ) = [ \frac{( Test Sample - Low Control )}{( High Control - Low Control )} ] \times 100 \%$$

Using cytotoxicity (%), it was determined that IDANPs posed 0.00% cytotoxicity to BEAS-2B and HeLa cell lines, and 4.27% cytotoxicity to the Vero cell line.

XTT Cytotoxicity Assay.

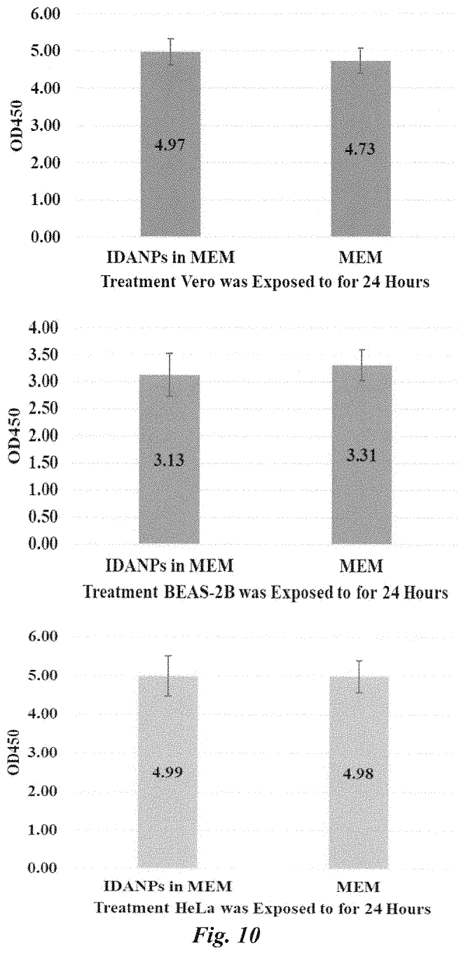

Vero, BEAS-2B, and HeLa cells were lifted from 75 cm² flasks with 5 mL trypsin (0.25%)-EDTA before being pelleted by centrifugation (3000 rpm, 5 min), and trypsin was subsequently replaced with MEM (10% FBS, 1% P-S). Cells were counted directly by hemocytometer and plated in a 96-well plate (7,000-9,000 cells per well) in 100 μL MEM (10% FBS, 1% P-S) and grown to confluence over the next 1-2 days. After cells were confluent, MEM was removed from the wells and replaced with either: (1) fresh MEM (11 wells), (2) fresh MEM with suspended IDANPs (25° C., 30% Fe, 5.5 mM citrate at 1.54 mg/mL, 11 flasks), Dulbecco's phosphate buffered saline (DPBS, 2 flasks) as a vehicle control. Treatments were applied to cell monolayers for 24 hr. During the last hour of exposure to the treatments, XTT was dissolved 0.01 g into 10 mL DPBS, and phenazine methosulfate (PMS) was dissolved 0.05 g into 1 mL sterile, deionized water (18MΩ). Then, 100 μL PMS solution was pipetted into 5 mL of the XTT solution. During preparation, all of these solutions were kept on ice. In addition, 10 μL cell lysis solution was added to one well containing MEM only, and one well containing MEM with suspended IDANPs as dead controls. At the conclusion of treatment, all solution was removed from the wells, cell monolayers were washed 2× with DPBS, and replaced with fresh MEM. This wash/replacement procedure was used to eliminate any signal coming from XTT interacting with IDANPs or IDANPs alone. Each well was subsequently inoculated with 100 μl XTT/PMS solution, and plates were placed back into the incubator for 2 hrs, with final absorption read at 450 nm (reference at 620 nm). During these studies, 10 replicate wells were treated with MEM only, ten with MEM with suspended IDANPs, two dead controls (one per MEM or MEM with IDANPs) were used, as well as two vehicle controls (treated with DPBS). Statistics were evaluated in SigmaPlot using a one way ANOVA V.11, and it was determined that no significant difference in mitochondrial enzyme activity occurred between Vero, BEAS-2B, or HeLa cells which had or had not been exposed to IDANPs (FIG. 10).

Plaque Assay: IDANP Influence Over HSV-1 Infection of Vero Cell Line.

In plaque assays, HSV-1 infection of Vero cells was carried out in 199V viral media (Blaho et al., Current Protocols in Microbiology, 2005) with (199V^{NP}) or without (199V) suspended IDANPs. Three separate plaque assay experiments were performed, with four, five, and seven pseudo-replicates per experimental treatment (199V or 199V^{NP}), as well as two negative control flasks which contained 199V or 199V^{NP} without HSV-1. Results from the three plaque assays were standardized by dividing each 199V^{NP}-HSV-1 plaque count with the average control (199V-HSV-1) plaque count for that particular plaque assay. When summarized, results show an 84% decrease in plaques (p<0.001) from Vero cell monolayers treated with 199V^{NP}-HSV-1 versus Vero cell monolayers treated with the control, 199V-HSV-1 (FIG. 8).

Plaque Assay: IDANP Influence Over HSV-1 Infection of BEAS-2B Cell Line.

In plaque assays, HSV-1 infection of BEAS-2B cells was carried out in 199V viral media (Blaho et al., Current Protocols in Microbiology, 2005) with (199V^{NP}) or without (199V) suspended IDANPs. Three separate plaque assay experiments were performed, with three, six, and seven pseudo-replicates per experimental treatment (199V or 199V^{NP}), as well as two negative control flasks which contained 199V or 199V^{NP} without HSV-1. Results from the three plaque assays were standardized by dividing each 199V^{NP}-HSV-1 plaque count with the average control (199V-HSV-1) plaque count for that particular plaque assay. When summarized, results show a 71% decrease in plaques (p<0.001) from BEAS-2B cell monolayers treated with 199V^{NP}-HSV-1 versus Vero cell monolayers treated with the control, 199V-HSV-1 (FIG. 9). Additional plaque assays were also performed to determine if lower dosage of IDANP could be used to still effectively prevent HSV-1 infection of BEAS-2B cells. Plaque assay results showed IDANPs diluted 10× (to 0.154 mg/mL) in 199V media still influenced a 28% decrease in HSV-1 infection of BEAS-2B cell monolayers. IDANPs diluted beyond 12× or more did not affect HSV-1 infection of BEAS-2B as compared to the control (FIG. 7).

LDH Cytotoxicity Assays: Cytotoxicity Posed by IDANPs in Vero, BEAS-2B, and HeLa Cell Lines.

LDH cytotoxicity assay performed included a 24 hr. exposure period, in which IDANPs were exposed to Vero, BEAS-2B, or HeLa cell monolayers in the same concentration as was delivered during plaque assay experiments (1.54 mg/mL). IDANPs imposed 4.27% cytotoxicity in the Vero cell line, and 0.00% cytotoxicity in the BEAS-2B and HeLa cell lines.

XTT Cytotoxicity Assays: Cytotoxicity Posed by IDANPs in Vero, BEAS-2B, and HeLa Cell Lines.

LDH cytotoxicity assay performed included a 24 hr. exposure period, in which, IDANPs suspended in MEM (10% FBS, 1% P-S) were exposed to Vero, BEAS-2B, or HeLa cell monolayers in the same concentration as was delivered during plaque assay experiments (1.54 mg/mL). In all cases, IDANPs did not cause statistically significant decreases in enzyme activity in Vero, BEAS-2B, or HeLa cell lines (FIG. 10). Statistics were evaluated in SigmaPlot V.11 using a one-way ANOVA. Listed are the corresponding p-values for each XTT experiment comparing mammalian cell lines exposed or not exposed to IDANPs: Vero (p=0.276), BEAS-2B (0.131), and HeLa (p=0.960).

IDANPs have demonstrated a unique influence over phage infection and killing of bacteria cells, in which IDANP-exposed bacterial cultures experience up to 2× the bacterial death as compared to controls (Andriolo et al., Journal of Vacuum Science and Technology B, 2013). As antibacterial resistance to mainstream antibiotics increases (Centers for Disease Control and Prevention, 2013), phage have been suggested as an alternative antibiotic therapy. IDANPs are composed of HA, a material found in mammalian bones and teeth and used in many FDA approved medical applications (Palmer et al., Chemical Reviews, 2008 & Hench, Journal of the American Ceramic Society, 1998). The potential biocompatibility of IDANP's, coupled with the functionality of these NPs as an aid to an alternative antibiotic therapy, make them of interest for medical applications. Here, IDANPs were examined in mammalian systems to ensure IDANP adjuvants used to increase phage infection of bacteria would not also increase mammalian viral infection in a mammalian system. Results of plaque assay studies in both Vero and BEAS-2B cell lines show that IDANPs, do not increase HSV-1 infection, but rather decrease HSV-1 infection of Vero cells by 84% and BEAS-2B cells by 71%. The observed therapeutic potential of IDANPs garnered from our plaque assay studies prompted a cytotoxicity evaluation of IDANPs using LDH and XTT cytotoxicity assays in Vero, BEAS-2B, and HeLa cell lines. Cytotoxicity results show IDANPs are largely non-toxic to Vero, BEAS-2B, and HeLa cell lines. Previous work has shown iron(III) inactivates HSV-1 (Sagripanti et al., Applied and Environmental Microbiology, 1993), and in more recent studies, it has been shown that iron(III) inhibits replication of DNA and RNAviruses (Terpilowska et al., Biometals, 2017). However, in the latter publication (Terpilowska et al., Biometals, 2017), a MTT cytotoxicity evaluation is provided, which shows that at approximately 150 μM (as is found in IDANPs), iron caused a reduction in cell viability of HEp-2 (HeLa contaminant) cells to ˜73% (Terpilowska et al., Biometals, 2017). For comparison, at the same concentration, IDANPs maintain 100% cell viability at 150 μM in Vero and HeLa cell lines, and 94.6% in the BEAS-2B cell line. These findings suggest that the HA matrix of the IDANP provides a biocompatible method/mechanism for iron delivery, and that IDANPs act as an effective and safe anti-viral agent.

In Vivo Mouse Studies.

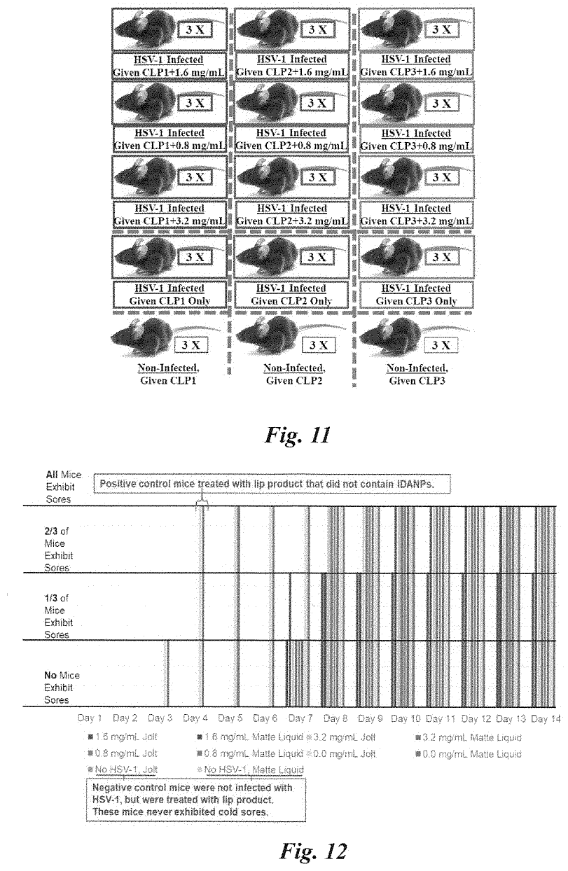

To demonstrate feasibility of IDANPs to not only prevent viral infection and replication, but to also prevent signs and symptoms associated with viral infection and replication, IDANP formulations were tested in vivo in mice at ARC at MSU. Protocols and euthanasia stipulations and methods were pre-approved by the biosafety and institutional animal care and use committees at ARC at MSU. To prepare treatments, IDANPs were mixed by hand into three different CLPs at three different concentrations (0.8 mg/mL, 1.6 mg/mL, and 3.2 mg/mL). The two CLPs used that allowed observable sign and/or symptom formation were both distributed by Maybelline®. Lip Studio™ Color Jolt™ Intense Lip Paint contained polybutene, pentaerythrityl tetraisostearate, propylene carbonate, microcrystalline wax, and alumina viscosity modifiers, while Color Sensational® Vivid Matte Liquid™ Lipstick contained dimethicone, dimethicone crosspolymer, hydrogenated polyisobutene, polyethylene, vinyl dimethicone/methicone silsesquioxane crosspolymer. Mice were infected with HSV-1 by a simple abrading of the lip epithelium by scapula and painting on of HSV-1 (1×10⁸ pfu/mL). Mice were thereafter treated three times per day with treatment for five days and observed post-treatment by laboratory technicians. Experimental plan is depicted in FIG. 11. The following treatments were each given to three mice so that statistical significance of the results were valid: (1) CLP 1+0.8 mg/mL IDANPs, (2) CLP 1+0.16 mg/mL IDANPs, (3) CLP 1+3.2 mg/mL IDANPs, (4) CLP 2+0.8 mg/mL IDANPs, (5) CLP 2+0.16 mg/mL IDANPs, (6) CLP 2+3.2 mg/mL IDANPs, (7) CLP 3+0.8 mg/mL IDANPs, (8) CLP 3+0.16 mg/mL IDANPs, (9) CLP 3+3.2 mg/mL IDANPs, (10) CLP 1, (11) CLP 2, (12) CLP 3. In addition, treatments (11), (12), and (13) were also given to three mice each, who were not infected with HSV-1 to serve as negative controls. As stated previously, one of the CLPs stained mice lips in such a way that signs and/or symptoms were not observable by the technicians. Mice that exhibited significant swelling, redness, or lesion formation, were humanely euthanized. Daily logs taken by laboratory technicians were used to create the graph of results shown in FIG. 12. All mice infected with HSV-1 and treated with CLPs NOT containing IDANPs exhibited lesion formation three days after being infected with HSV-1 and were euthanized. All mice infected with HSV-1 and treated with one of the two CLPs CONTAINING ANY CONCENTRATION of IDANPs did not exhibit swelling, redness, or lesion formation which warranted euthanasia for the five-day treatment period, plus one day where the mice were not treated. Most mice were euthanized on the eighth day post-HSV-1 infection (three days after treatment ceased). However, one mouse did not exhibit symptoms until the ninth day (four days post infection), and one mouse did not exhibit symptoms until day fourteen (nine days post infection). Results of this study demonstrate that IDANPs not only prevent signs and symptoms associated with viral infection during treatment, but that IDANP treatment prevents future signs and symptoms associated with viral infection even after treatment has ceased.

Active Ingredient Studies.

Using plaque assays, IDANP ingredients were used individually to determine the active ingredient that caused the decrease in mammalian cell death by virus. The most telling result from those studies occurred when apatite nanoparticles (ANPs) made with identical synthesis methods but excluding iron were used. In those studies, the average plaque count associated with Vero cell monolayers exposed to HSV-1 simultaneously with ANPs was 42 pfu/mL, while Vero cell monolayers exposed to HSV-1 simultaneously with IDANPs was 9.3 pfu/mL. This study clearly showed that iron was required for diminished mammalian cell death by HSV-1 and was therefore identified as the active ingredient in IDANPs.

Minimum Effective Dosage.

The half maximal inhibitor concentration (IC₅₀) of IDANPs was evaluated by diluting IDANPs from 1.60 mg/mL to 0.80 (1×), 0.32 (2×), 0.23 (7×), and 0.16 mg/mL during plaque assays. At IC₅₀, IDANP concentration was 0.23 mg/mL, and HSV-mediated Vero cell death was decreased by 40%.

Mechanism Studies.

To determine if IDANPs interrupted viral replication, flasks containing Vero cell monolayers were inoculated with the same dosage of HSV-1, 12 flasks with and 12 flasks without IDANPs. Plaque assay methods were then carried out as usual. After 3 days post-infection, the amount of HSV-1 DNA present was quantified using qPCR. While the average quantitation cycle (Cq*) for Vero cells exposed to only HSV-1 during infection was 12.35, Vero cells exposed to HSV-1 and suspended IDANPs (1.60 mg/mL) was 22.4, indicating a significant reduction in viral replication had occurred in the IDANP-exposed flasks. In a second study, Vero cell monolayers were exposed to HSV-1 with or without IDANPs and allowed adequate time for viral attachment and penetration into host cells (2 hr) but not enough time for host cell lysis to occur (3 days). At 2 hr, infected Vero cells were washed 3× to remove noninternalized or attached viruses. Cells were then immediately harvested and DNA isolated. HSV-1 DNA was quantified by real-time polymerase chain reaction (qPCR), and HSV-1 infection alone was compared to HSV-1 infection with IDANPs. No statistically significant difference (p=0.986) in the amount of HSV-1 DNA present was observed, with the Cq for HSV-1-alone replicates being an average of 27.2 (Std. Dev.=1.7), and HSV-1 with IDANPs being an average of 27.16 (Std. Dev.=2.2). Collectively, active ingredient and preliminary mechanistic studies point to an iron-mediated mechanism that interrupts HSV-1 replication intracellularly at a point after entry. *Cq is the cycle number at which a detectable amount of fluorescence is achieved, and is the basic result of qPCR. The qPCR process occurs in a number of cycles, in which, genetic material is replicated and detected by fluorescence. The cycle number at which there is enough genetic material for detection is the Cq value. Generally, lower Cq values mean higher initial copy numbers of the target genetic material because it took less time for the genetic material to replicate to high enough levels for detection. In the first qPCR study, IDANP exposure increased the Cq value from 12.35 to 22.4, meaning decreased the amount of viral DNA present. This shows IDANPs prevent viral DNA replication. In the second qPCR study, we looked only at the amount of viral DNA that had gotten into the mammalian host cells. In this way, we could determine if prevention of viral replication was occurring before or after viral DNA had entered the mammalian host cell. There was no statistically significant difference in the amount of viral DNA inside host mammalian cells. Collectively, these studies show that IDANPs are not preventing viral DNA from entering mammalian host cells, and are interrupting viral replication at some point after viral DNA has entered the host cells.

As such, compositions and methods of using IDANPs for the prevention, treatment, or alleviation of signs or symptoms associated with viral activation or infection are disclosed herein. In one embodiment, IDANPs are suspended in any medium suitable for therapeutic delivery to virus infected or affected cells. One exemplary embodiment includes suspension of IDANPs in a semi-solid medium capable of direct application to virus infected or affected tissue, such as lips, similar to how chap stick is applied. Other exemplary embodiments include IDANPs suspended in medium consisting of one of the following: solid, semi-solid, Newtonian or Non-Newtonian fluid, or powder.

IDANP compositions used for prevention, treatment, or alleviation of signs or symptoms associated with viral activation or infection are prepared as follows: IDANPs are synthesized as stated herein (see [0007] and [0026]). In one embodiment, supernatant created during IDANP synthesis/preparation is removed by centrifugation of the particles and removal of supernatant by pipette. IDANPs without supernatant are then suspended by any suitable means into any medium suitable for therapeutic delivery to virus infected or affected cells. In this embodiment, suspending the IDANPs is accomplished by mixing IDANPs into the selected media by stir bar on a stir plate, or vortexing. One skilled in the art would recognize that various suspension means could be utilized to prepare IDANPs for delivery.

Once the IDANPs are suspended in the selected medium, the medium is delivered to the virus affected or infected cells by any accepted therapeutic means. One skilled in the art would recognize that such compositions could be delivered by various therapeutic means including, but not limited to injection, oral administration, or direct application. In an exemplary embodiment, the semi-solid IDANP medium would be delivered by direct application to the lips, similar to the application of chap stick, to treat signs or symptoms associated with viral activation or infection

It is understood that the foregoing specific examples are merely illustrative of the present invention. Certain modifications of the compositions and/or methods may be made and still achieve the objectives of the invention. Such modifications are contemplated within the scope of the claimed invention.

What is claimed is:

1. A composition for the prevention, treatment, or alleviation of signs or symptoms associated with viral activation, infection of, or replication in mammalian cells comprising functionalized iron-doped apatite nanoparticles (IDANPs).

2. The composition of claim 1 where said functionalized IDANPs are further comprised of citrate functionalized IDANPs.

3. The composition of claim 1 where said functionalized IDANPs are delivered to cells or tissues affected or infected by a mammalian virus or viruses.

4. The composition of claim 1 where said functionalized IDANPs are delivered to cells or tissues exhibiting signs or symptoms caused by mammalian viral infection, activation, or disease.

5. The composition of claim 1 where said functionalized IDANPs are suspended in a medium.

6. The composition of claim 5 where said medium is delivered to cells or tissues exhibiting signs or symptoms caused by mammalian viral infection, activation, or disease.

7. The composition of claim 5 where said medium is selected from the group consisting of: solid, semi-solid, Newtonian fluid, Non-Newtonian fluid, and powder.

8. The composition of claim 5 where said medium is delivered by therapeutic means including injection, oral administration, absorption, or direct application.

9. The composition of claim 1 where said composition decreases viral infection, activation, or replication.

10. The composition of claim 9 where said functionalized IDANPs are further comprised of citrate functionalized IDANPs.

11. The composition of claim 9 where said functionalized IDANPs are delivered to cells or tissues affected or infected by a mammalian virus or viruses.

12. The composition of claim 9 where said functionalized IDANPs are delivered to cells or tissues exhibiting signs or symptoms caused by mammalian viral infection, activation, or disease.

13. The composition of claim 9 where said functionalized IDANPs are suspended in a medium.

14. The composition of claim 13 where said medium is delivered to virus affected or infected mammalian cells.

15. The composition of claim 13 where said medium is selected from the group consisting of: solid, semi-solid, Newtonian fluid, Non-Newtonian fluid, and powder.

16. The composition of claim 13 where said medium is delivered by therapeutic means including injection, oral administration, absorption, or direct application.

17. The composition of claim 1 where said functionalized IDANPs are delivered to cells or tissues prior to being affected or infected by a mammalian virus or viruses.

18. The composition of claim 1 where said functionalized IDANPs are delivered to cells or tissues prior to occurrence of signs or symptoms caused by mammalian viral infection, activation, or disease.
